(19) United States
(12) Patent Application Publication
(10) Pub. No.: US 2024/0238574 A1
(43) Pub. Date: Jul. 18, 2024

(54) Low-Profile Transitionable Access Port

(71) Applicant: Bard Peripheral Vascular, Inc., Franklin Lakes, NJ (US)

(72) Inventors: Jessica Hoye, Phoenix, AZ (US); Bryon Ray Densley, Rochester, MN (US); Christian Andersen, Queen Creek, AZ (US); Diana Fiumefreddo, Salt Lake City, UT (US); Ian N. Thomas, West Bountiful, UT (US)

(21) Appl. No.: 18/561,945

(22) PCT Filed: May 20, 2021
(86) PCT No.: PCT/US21/33386

§ 371 Date: Nov. 17, 2023

(51) Int. Cl. A61M 39/02; A61M 39/04

(52) U.S. Cl. CPC A61M 39/0208; A61M 39/04; A61M 2039/0232; A61M 2207/10

(57) ABSTRACT

Embodiments disclosed herein are directed to a port system including a body defining a reservoir and a stem. One of the body can include a lower housing and an upper housing slidably engaged therewith and be transitionable between an expanded configuration and a collapsed configuration. The port body in the collapsed configuration can define a smaller volume or outer profile than the port body in the expanded configuration. The reservoir in the collapsed configuration can define a smaller volume than the reservoir in the expanded configuration. A biasing member can bias the port to the expanded configuration. A latching mechanism can retain the port in one of the expanded configuration or the collapsed configuration. An actuator can transition latching mechanism between a locked position and an unlocked position. Subcutaneous placement of the port in the collapsed configuration can require a smaller incision site, fewer stitches and improved patient recovery times.

### SUMMARY

Briefly summarized, embodiments disclosed herein are directed to a subcutaneous access port transitionable between an expanded configuration and a collapsed configuration, and associated methods thereof. The port can include a body having a lower housing and an upper housing slidably engaged therewith. The lower housing and the upper housing can co-operate to define a reservoir. In the expanded configuration the port can define a first port volume and a first reservoir volume. In the collapsed configuration the port can define a second port volume that is less than the first port volume, and a second reservoir volume that is less than the first reservoir volume. Advantageously, the port can transition to the collapsed configuration to provide a reduced overall size or outer profile for insertion. Optionally the port can provide a reduced overall size between access events. As such, the port can require a smaller incision site, require fewer stitches or no stitches at all, improving patient recovery times, patient comfort, reducing scarring and improving aesthetics.

Disclosed herein is a subcutaneous access port including, a body having a lower housing and an upper housing slidably engaged therewith and co-operating to define a reservoir, the body transitionable between an expanded configuration and a collapsed configuration, the collapsed configuration defining a smaller overall profile, a needle penetrable septum disposed in a reservoir opening disposed in a top surface of the upper housing and configured to provide percutaneous access thereto, and a port stem defining a stem lumen in fluid communication with the reservoir.

In some embodiments, the port body in the expanded configuration defines a first port height, and wherein the port body in the collapsed configuration defines a second port height, the second port height being less than the first port height. In some embodiments, the port body in the expanded configuration defines a first port volume, and the port body in the collapsed configuration defines a second port volume, the second port volume being less than the first port volume. In some embodiments, the reservoir in the expanded configuration defines a first reservoir height, and the reservoir in the collapsed configuration defines a second reservoir height, the second reservoir height being less than the first reservoir height.

In some embodiments, the reservoir in the expanded configuration defines a first reservoir volume, and the reservoir in the collapsed configuration defines a second reservoir volume, the second reservoir volume being less than the first reservoir volume. In some embodiments, the second reservoir volume defines a zero volume or a de minimis volume. In some embodiments, the lower housing is telescopically engaged with the upper housing and slidably engaged to transition between the expanded configuration and the collapsed configuration. In some embodiments, an outer surface of the upper housing is slidably engaged with an inner surface of the lower housing. In some embodiments, an inner surface of the upper housing is slidably engaged with an outer surface of the lower housing. In some embodiments, one of the upper housing or the septum occludes the stem lumen when the port is in the collapsed configuration.

In some embodiments, the subcutaneous access port further includes a biasing member configured to bias the port towards the expanded configuration. In some embodiments, the subcutaneous access port further includes a latching mechanism transitionable between a locked position and an unlocked position, and configured to retain the upper housing in one of the expanded configuration or the collapsed configuration. In some embodiments, the latching mechanism is configured to lock the upper housing in an expanded configuration when a needle accesses the reservoir. In some embodiments, the latching mechanism is configured to engage and retain the upper housing in a collapsed configuration and disengage the upper housing when a top surface of the port is depressed.

In some embodiments, the subcutaneous access port further includes an actuator coupled to the latching mechanism and configured to transition the latching mechanism between a locked position and an unlocked position. In some embodiments, the actuator is one of a push button, slider, or rocker switch disposed on one of a top surface or a side surface of the body. In some embodiments, the actuator includes a magnetic switch configured to transition the latching mechanism between a locked position and an unlocked position when a magnetic having a magnetic field is placed proximate the actuator. In some embodiments, the actuator is configured to receive a signal to transition the latching mechanism between a locked position and an unlocked position, the signal including one of a magnetic, electro-magnetic, radio-frequency, acoustic, or optical modality signal.

Also disclosed is a method of manufacturing a subcutaneous access port including, forming a port body having a lower housing and an upper housing slidably engaged therewith and co-operating to define a reservoir, the body transitionable between an expanded configuration and a collapsed configuration, the collapsed configuration defining a smaller overall profile, coupling a needle penetrable septum disposed in a reservoir opening disposed in a top surface of the upper housing and configured to provide percutaneous access thereto, and forming a port stem coupled to the body and defining a stem lumen in fluid communication with the reservoir.

In some embodiments, the port body in the expanded configuration defines a first port height, and wherein the port body in the collapsed configuration defines a second port height, the second port height being less than the first port height. In some embodiments, the port body in the expanded configuration defines a first port volume, and the port body in the collapsed configuration defines a second port volume, the second port volume being less than the first port volume. In some embodiments, the reservoir in the expanded configuration defines a first reservoir height, and the reservoir in the collapsed configuration defines a second reservoir height, the second reservoir height being less than the first reservoir height.

In some embodiments, the reservoir in the expanded configuration defines a first reservoir volume, and the reservoir in the collapsed configuration defines a second reservoir volume, the second reservoir volume being less than the first reservoir volume. In some embodiments, the second reservoir volume defines a zero volume or a de minimis volume. In some embodiments, the method further includes telescopically engaging the lower housing with the upper housing to slidably transition between the expanded configuration and the collapsed configuration. In some embodiments, an outer surface of the upper housing is slidably engaged with an inner surface of the lower housing.

In some embodiments, an inner surface of the upper housing is slidably engaged with an outer surface of the lower housing. In some embodiments, one of the upper housing or the septum occludes the stem lumen when the port is in the collapsed configuration. In some embodiments, the method further includes forming a biasing member within the port body configured to bias the port towards the expanded configuration. In some embodiments, the method further includes a latching mechanism transitionable between a locked position and an unlocked position, and configured to retain the upper housing in one of the expanded configuration or the collapsed configuration.

In some embodiments, the latching mechanism is configured to lock the upper housing in an expanded configuration when a needle accesses the reservoir. In some embodiments, the latching mechanism is configured to engage and retain the upper housing in a collapsed configuration and disengage the upper housing when a top surface of the port is depressed. In some embodiments, the method further includes an actuator coupled to the latching mechanism and configured to transition the latching mechanism between a locked position and an unlocked position.

In some embodiments, the actuator is one of a push button, slider, or rocker switch disposed on one of a top surface or a side surface of the body. In some embodiments, the actuator includes a magnetic switch configured to transition the latching mechanism between a locked position and an unlocked position when a magnetic having a magnetic field is placed proximate the actuator. In some embodiments, the actuator is configured to receive a signal to transition the latching mechanism between a locked position and an unlocked position, the signal including one of a magnetic, electro-magnetic, radio-frequency, acoustic, or optical modality signal.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a port coupled to a catheter, in accordance with embodiments disclosed herein.

FIG. 2A shows a longitudinal cross-section view of the port in an expanded configuration, in accordance with embodiments disclosed herein.

FIG. 2B shows a longitudinal cross-section view of the port in a collapsed configuration, in accordance with embodiments disclosed herein.

FIG. 3A shows a lateral cross-section view of the port in an expanded configuration, in accordance with embodiments disclosed herein.

FIG. 3B shows a lateral cross-section view of the port in a collapsed configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

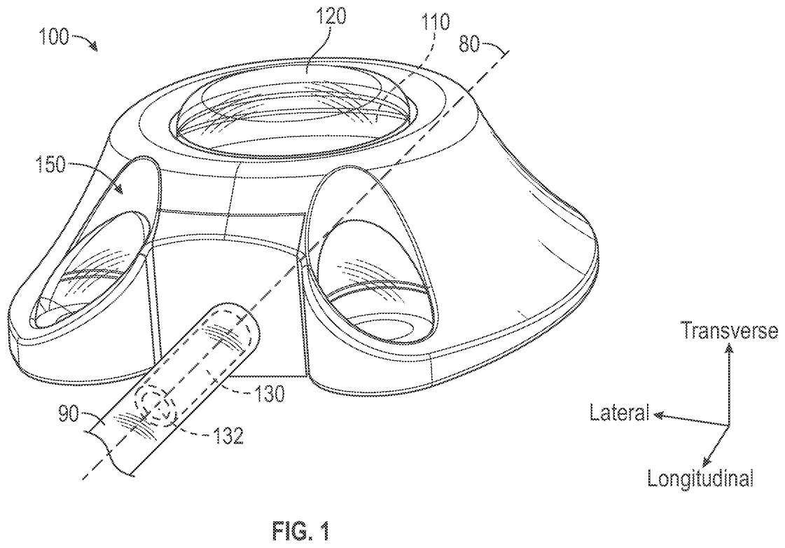

To assist in the description of embodiments described herein, as shown in FIG. 1, a longitudinal axis extends substantially parallel to an axial length of the port stem. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

FIG. 1 shows a vascular access device, or “port” 100 configured for subcutaneous placement. The port 100 can generally include a body 150 defining a reservoir 110 and including a needle penetrable septum 120 thereover. The port 100 can further include a port stem 130, defining a stem lumen 132, and is in fluid communication with the reservoir 110. The port stem 130 can engage a lumen of a catheter 90 in an interference fit, to provide fluid communication therewith. A distal tip of the catheter 90 can be disposed within the vasculature of a patient. In use, an access needle can penetrate the skin surface and access the reservoir by piercing the septum 120. Fluids can then be disposed within the reservoir 100, or withdrawn from the reservoir 110.

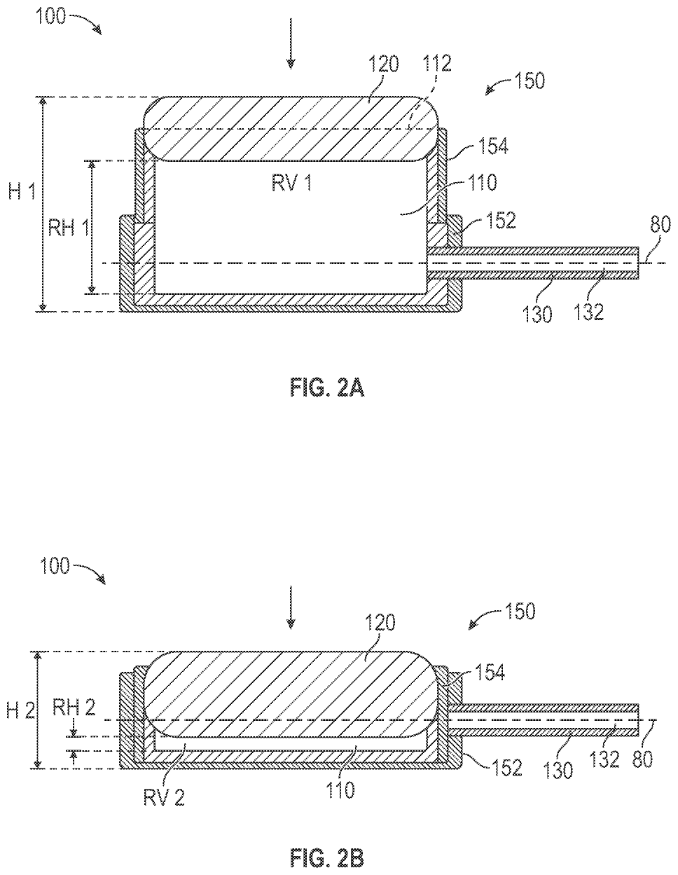

In an embodiment, as shown in FIGS. 2A-2B, the port 100 can include a port body 150 including a first housing, or lower housing 152, and a second housing, or an upper housing 154, slidably engaged therewith. The lower housing 152 and the upper housing 154 can co-operate to define the reservoir 110. In an embodiment, the upper housing 154 can define a reservoir opening 112 disposed in a top surface thereof and configured to retain the needle-penetrable septum 120 therein. In an embodiment, the septum 120 can be formed of a silicone rubber or similar suitable material. In use, the reservoir 110 can be accessed percutaneously by an access needle, penetrating the septum 120 and fluidly accessing the reservoir 110.

In an embodiment, the port 100 can further include a port stem 130 extending along an axis 80. The stem 130 can define a stem lumen 132 that is in fluid communication with the reservoir 110. The stem 130 can be configured to be coupled to a catheter 90 or similar device, configured to access a vasculature of a patient. The catheter 90 can include an elongate body defining a lumen 92 extending therethrough. In an embodiment, the port body 150 can be formed of a substantially rigid material including plastic, polymer, metal, alloy, composite, or the like.

In an embodiment, the port 100 can be transitionable between an expanded configuration (FIG. 2A) and a collapsed configuration (FIG. 2B) to facilitate subcutaneous placement. In an embodiment, the lower housing 152 and the upper housing 154 can be slidably engaged with each other and can transition between a first, or expanded, configuration (FIG. 2A) and a second, or collapsed configuration (FIG. 2B). In an embodiment, an outer surface of the upper housing 154 can be telescopically engaged with an inner surface of the lower housing 152. In an embodiment, an inner surface of the upper housing 154 can be telescopically engaged with an outer surface of the lower housing 152.

In an embodiment, in the expanded configuration, the port 100 can define a first port height (H1). In an embodiment, in the expanded configuration, the reservoir 110 can define a first reservoir height (RH1). In an embodiment, in the collapsed configuration, the port 100 can define a second port height (H2). The second port height (H2) can be less than the first port height (H1). In an embodiment, in the collapsed configuration, the reservoir 110 can define a second reservoir height (RH1). The second reservoir height (RH2) being less than the first reservoir height (RH1).

In an embodiment, in the expanded configuration, the port 100 can define a first port volume (VI). In an embodiment, in the collapsed configuration, the port 100 can define a second port volume (12). The second port volume (12) being less than the first port volume (VI). In an embodiment, in the expanded configuration, the reservoir 110 can define a first reservoir volume (RV1). In an embodiment, in the collapsed configuration, the reservoir 110 can define a second reservoir volume (RV2). The second reservoir volume (RV2) being less than the first reservoir volume (RV1). In an embodiment, the second reservoir volume (RV2) can define a zero volume or a de minimis volume.

In an embodiment, a portion of the septum 120 or a portion of the body 150, e.g. a portion of the lower housing 152 or a portion of the upper housing 154, can occlude the port stem lumen 132 when the port 100 is in the collapsed configuration. As such, fluid communication between the reservoir 110 and the catheter lumen 92, coupled to the port stem 130 can be inhibited.

In an embodiment, the port 100 can include a biasing member (e.g. spring or the like) configured to bias the port towards the expanded configuration. In an embodiment, the port 100 can include a latch mechanism configured to selectively retain the port 100 in one of the expanded or collapsed configurations. In use, a clinician can selectively transition the port 100 between the expanded configuration and the collapsed configuration.

In an embodiment, as shown in FIGS. 2A-2B, a clinician can depress the top surface of the port 100, e.g. a top surface of the septum 120 or a top surface of the upper housing 154, to overcome the force of the biasing member and slidably transition the upper housing 154 from the expanded configuration (FIG. 2A) to the collapsed configuration (FIG. 2B). Once in the collapsed configuration, the latch mechanism can engage the upper housing 154 to retain the upper housing 154 in the collapsed configuration.

In an embodiment, the clinician can selectively transition the port 100 from the collapsed configuration to the expanded configuration. The clinician can depress the top surface of the port 100, e.g. a top surface of the septum 120 or a top surface of the upper housing 154, to overcome the force of the biasing member and disengage the latching mechanism. The clinician can then release the top surface of the port 100 and allow the biasing member to transition the upper housing 154 to the expanded configuration.

In an embodiment, the force of the biasing member can maintain the port 100 in the expanded configuration. In an embodiment, the force of the biasing member can maintain the port 100 in the expanded configuration even when the access needle is urged through the needle-penetrable septum 120 to access the reservoir 110 therebelow. In an embodiment, the latching mechanism can be configured to maintain the port 100 in the expanded configuration. As such, a clinician can urge the access needle through the septum 120 without depressing the top surface of the port 100 and transitioning the port from the expanded configuration to the collapsed configuration unintentionally.

In an embodiment, the latching mechanism can be transitioned between a locked position and an unlocked position. In the locked position the latching mechanism is prevented from disengaging the upper housing 154. In the unlocked position, the latching mechanism can disengage the upper housing 154 allowing the port 100 to transition between the expanded configuration and the collapsed configuration. In an embodiment, the latching mechanism can be maintained in a locked position by the presence of the needle accessing the reservoir 101. For example, the needle accessing the reservoir 110 can engage the latching mechanism maintain the latching mechanism in the locked position, preventing the port 100 from transitioning between the expanded configuration and the collapsed configuration. Advantageously, the latching mechanism can prevent accidental transition of the port 100 between the expanded configuration and the collapsed configuration, for example, when the port 100 is in use, and/or when a needle is engaged with the reservoir 110.

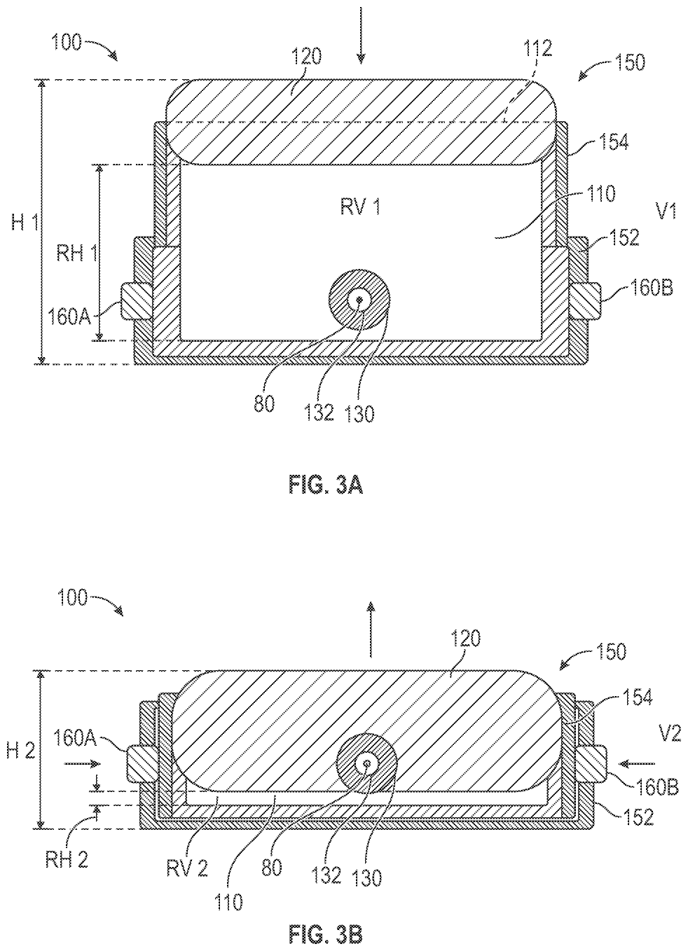

In an embodiment, as shown in FIGS. 3A-3B, the port 100 can include an actuator 160 coupled to the latching mechanism and configured to disengage the latching mechanism and/or transition the latching mechanism between the locked position and the unlocked position. In use, a clinician can transition the port 100 to the collapsed configuration by depressing the top surface of the port 100, as described herein (FIG. 3A). The latching mechanism can engage the upper housing 154 and retain the upper housing 154 in the collapsed configuration. The clinician can then actuate the actuator 160 to disengage the upper housing 154. The biasing member can then transition the upper housing to the expanded configuration (FIG. 3B).

In an embodiment, the latching mechanism can engage the upper housing 154 and maintain the upper housing 154 in the expanded configuration. In use, a clinician can actuate the actuator 160 to disengage the latching mechanism and allow the upper housing 154 to transition from the expanded configuration to the collapsed configuration. Advantageously, the actuator 160 and latching mechanism can be configured to prevent accidental transition of the port 100 from the expanded configuration to the collapsed configuration, for example, when the port 100 is in use and/or when a needle is engaged with the reservoir 110.

In an embodiment, the actuator 160 can be a push button, slider, rocker switch, or similar mechanical actuator disposed on an outer surface of the port 100, e.g. a top surface, side surface, etc. In an embodiment, the port 100 can include a first actuator 160A disposed on a first side surface of the port, and a second actuator 160B disposed on a second side surface of the port 100, opposite the first side surface. The clinician can actuate one of the first actuator 160A or the second actuator 160B by applying a lateral “pinching” force to the port 100. Advantageously, the lateral pinching force and can mitigate applying pressure directly to the patient.

In an embodiment, the actuator 160 can include a magnetic switch configured to be transitioned between a first position and a second position by the presence or absence of a magnetic field. In use, with the port 100 placed subcutaneously, a clinician can position a magnet (permanent magnet or electro-magnet) proximate the port 100, e.g. on a skin surface adjacent the port. The presence of the magnetic field can actuate the actuator 160 to disengage the latching mechanism and allow the port to transition between the expanded configuration and the collapsed configuration.

In an embodiment, the actuator 160 can be actuated by a signal from one or more modalities, e.g. magnetic, electro-magnetic, radio-frequency, acoustic, optical, or the like. For example, the actuator 160 can be actuated percutaneously by sending or receiving a signal to the port 100 from a device disposed external to the patient. The actuator 160 can disengage the latching mechanism and transition the port 100, as described herein.

Advantageously, the collapsed configuration can provide a smaller overall profile, size, or volume to the port 100. As such, the port 100 in the collapsed configuration can be disposed subcutaneously through a relatively smaller insertion site. Advantageously, the smaller insertion site can required fewer stitches, or no stitches, to close the insertion site leading to reduced scaring, improved recovery times, improved patient comfort and improved aesthetics. In an embodiment, the port 100 can be transitioned between the expanded configuration and the collapsed configuration by the clinician actuating the latching mechanism or actuator 160 percutaneously. When the port 100 has been disposed subcutaneously, the port 100 can be selectively transitioned from the collapsed (second) configuration to the expanded (first) configuration ready for use.

In an embodiment, the port 100 can be transitioned from the expanded configuration to the collapsed configuration outside of the patient, as described herein. Once placed subcutaneously, the port 100 can remain in the collapsed configuration. In use, a clinician can selectively transition the port 100 from the collapsed configuration to the expanded configuration ready for use. Once the treatment has been completed, the clinician can selectively transition the port 100 from the expanded configuration to the collapsed configuration. Advantageously, the port 100 can maintain the collapsed configuration after subcutaneous placement between uses providing a lower profile, reducing stretching of the skin, reducing scaring, and improving patient comfort and aesthetics. When the port 100 is required to be accessed the port 100 can be selectively transitioned to the expanded configuration by the clinician actuating the latching mechanism percutaneously.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous access port, comprising:
a body including a lower housing and an upper housing slidably engaged therewith and co-operating to define a reservoir, the body transitionable between an expanded configuration and a collapsed configuration, the collapsed configuration defining a smaller overall profile;
a needle penetrable septum disposed in a reservoir opening disposed in a top surface of the upper housing and configured to provide percutaneous access thereto; and
a port stem defining a stem lumen in fluid communication with the reservoir.

2. The subcutaneous access port according to claim 1, wherein the port body in the expanded configuration defines a first port height, and wherein the port body in the collapsed configuration defines a second port height, the second port height being less than the first port height.

3. The subcutaneous access port according to claim 1, wherein the port body in the expanded configuration defines a first port volume, and the port body in the collapsed configuration defines a second port volume, the second port volume being less than the first port volume.

4. The subcutaneous access port according to claim 1, wherein the reservoir in the expanded configuration defines a first reservoir height, and the reservoir in the collapsed configuration defines a second reservoir height, the second reservoir height being less than the first reservoir height.

5. The subcutaneous access port according to claim 1, wherein the reservoir in the expanded configuration defines a first reservoir volume, and the reservoir in the collapsed configuration defines a second reservoir volume, the second reservoir volume being less than the first reservoir volume.

6. The subcutaneous access port according to claim 5, wherein the second reservoir volume defines a zero volume or a de minimis volume.

7. The subcutaneous access port according to claim 1, wherein the lower housing is telescopically engaged with the upper housing and slidably engaged to transition between the expanded configuration and the collapsed configuration.

8. The subcutaneous access port according to claim 7, wherein an outer surface of the upper housing is slidably engaged with an inner surface of the lower housing.

9. The subcutaneous access port according to claim 7, wherein an inner surface of the upper housing is slidably engaged with an outer surface of the lower housing.

10. The subcutaneous access port according to claim 1, wherein one of the upper housing or the septum occludes the stem lumen when the port is in the collapsed configuration.

11. The subcutaneous access port according to claim 1, further including a biasing member configured to bias the port towards the expanded configuration.

12. The subcutaneous access port according to claim 1, further including a latching mechanism transitionable between a locked position and an unlocked position, and configured to retain the upper housing in one of the expanded configuration or the collapsed configuration.

13. The subcutaneous access port according to claim 12, wherein the latching mechanism is configured to lock the upper housing in an expanded configuration when a needle accesses the reservoir.

14. The subcutaneous access port according to claim 12, wherein the latching mechanism is configured to engage and retain the upper housing in a collapsed configuration and disengage the upper housing when a top surface of the port is depressed.

15. The subcutaneous access port according to claim 12, further including an actuator coupled to the latching mechanism and configured to transition the latching mechanism between a locked position and an unlocked position.

16. The subcutaneous access port according to claim 15, wherein the actuator is one of a push button, slider, or rocker switch disposed on one of a top surface or a side surface of the body.

17. The subcutaneous access port according to claim 15, wherein the actuator includes a magnetic switch configured to transition the latching mechanism between a locked position and an unlocked position when a magnetic having a magnetic field is placed proximate the actuator.

18. The subcutaneous access port according to claim 15, wherein the actuator is configured to receive a signal to transition the latching mechanism between a locked position and an unlocked position, the signal including one of a magnetic, electro-magnetic, radio-frequency, acoustic, or optical modality signal.

19-36. (canceled)
